SEQUENCE LISTING (4 sequences)

SEQ ID NO: 1 - DNA, 1242 bp, Lysinibacillus composti
  source 1..1242: mol_type = genomic DNA
atggcagaaaacctgaacttatttacgagcacccaggaggttgtgaaagaagcgctgaacaaactgggttatgatgaggcaatgtacgaactgctgaaagaaccgctgcgcctgctgaaagtgcgtattcctgtgaagatggacgatggcaccacacaggtgtttacgggttatcgcgcacaacattccgatgcagtaggtcccaccaaaggtggcgtgcgttttcatcctatggtttctgaagacgaagttaaagcactgagcatgtggatgaccctgaagtgcgggattgtagatctgccttatggtggtggtaaaggtggcattatttgtgatccgcgtcagatgagcatgggggaattagaacgtctgagccgtggatatgttcgggcaattagtcagattgttgggccgaccaaagatataccgggtccggatatgtttaccaatgcacaaattatggcatggatgatggatgagtatagccgtatggatgaatttaatagtccgggttttataaccggtaaacctctggtgctgggcggtagtaaagggcgtgatcgggcgacggcagaaggtgttacgattgttattcaggaggcagcaaaaaagagaaatatcgatatcaaaggtgcacgcgttgttattcaagggttcggtaatgccggcagttttttagcaaagtttatgagtgatctgggcgcgaaggttataggaataagtgatgcatacggggccctgcacgatccgaatggtttagatattgattatctgctggacagacgtgatagttttggtaccgttaccacgctgtttgaaaatacaattacgaatcaggagctgctggaactggattgtgatattctggtgccggccgcaattgagaatcagattacggcagaaaatgcacataatattaaggcaaccatagttgtggaagcagcgaacggcccaaccacctctgaagcaaccaaaattctgaccgaacgtggtattctgttagtgccagacgttttagcaagcgcaggtggggcaacagttagctactttgagtgggttcaaaataatatgggctattactgggaagaagaagaggttcaagaaaaactgtacaaaaaaatgtatgatagctttgaagcagtatatacaaccgcaaccacgcgcaatatagatatgcgtctggcagcgtatatggtgggagtgagaagaacagcagaagcgagccgtttccggggctgggtg

SEQ ID NO: 2 - protein, 414 aa, Lysinibacillus composti
MAENLNLFTSTQEVVKEALNKLGYDEAMYELLKEPLRLLKVRIPVKMDDGTTQVFTGYRAQHSDAVGPTKGGVRFHPMVSEDEVKALSMWMTLKCGIVDLPYGGGKGGIICDPRQMSMGELERLSRGYVRAISQIVGPTKDIPGPDMFTNAQIMAWMMDEYSRMDEFNSPGFITGKPLVLGGSKGRDRATAEGVTIVIQEAAKKRNIDIKGARVVIQGFGNAGSFLAKFMSDLGAKVIGISDAYGALHDPNGLDIDYLLDRRDSFGTVTTLFENTITNQELLELDCDILVPAAIENQITAENAHNIKATIVVEAANGPTTSEATKILTERGILLVPDVLASAGGATVSYFEWVQNNMGYYWEEEEVQEKLYKKMYDSFEAVYTTATTRNIDMRLAAYMVGVRRTAEASRFRGWV

SEQ ID NO: 3 - DNA, 1017 bp, Bacillus stearothermophilus
  source 1..1017: mol_type = genomic DNA
atgaaggcggctgtcgtagaacagttcaaagaaccgctgaaaatcaaagaagtcgagaaaccgaccatctcttatggcgaagttctggttcgtatcaaagcgtgtggtgtctgtcacaccgatctgcatgcagcccacggtgattggccggtcaaaccgaaactgccactgatcccgggtcacgagggtgttggtatcgttgaagaagtgggtccaggcgtcacccatctgaaggtaggtgatcgcgttggcatcccttggctgtatagcgcatgcggtcattgcgattattgcctgtccggtcaggaaactctgtgcgaacaccagaagaatgcgggttactctgttgatggtggctacgcagaatactgtcgcgcggcggcagactacgttgttaaaattccggacaacctgtccttcgaagaagctgctccgattttctgtgcgggtgtgaccacctacaaagcgctgaaggttactggtgccaaaccaggtgaatgggttgctatctacggtatcggtggcctgggtcacgttgctgttcagtatgccaaagctatgggtctgaacgtggttgcggtagacattggtgatgagaaactggagctggctaaagaactgggtgcagacctggtagtgaaccctctgaaggaagacgccgcgaaatttatgaaagaaaaagtcggtggcgttcacgccgcggttgtaactgctgtttccaaaccagcattccagtctgcatacaactccatccgtcgtggtggtgcttgtgttctggttggtctgccgccggaggaaatgccgatccctattttcgataccgttctgaacggtattaaaatcatcggttccatcgttggtactcgtaaggatctgcaggaagctctgcaatttgctgcggaaggtaaagtcaaaaccattattgaagttcagccgctggagaaaattaacgaagttttcgaccgcatgctgaaaggtcagatcaacggccgtgttgtcctgacgctggaagacaaa

SEQ ID NO: 4 - protein, 339 aa, Bacillus stearothermophilus
MKAAVVEQFKEPLKIKEVEKPTISYGEVLVRIKACGVCHTDLHAAHGDWPVKPKLPLIPGHEGVGIVEEVGPGVTHLKVGDRVGIPWLYSACGHCDYCLSGQETLCEHQKNAGYSVDGGYAEYCRAAADYVVKIPDNLSFEEAAPIFCAGVTTYKALKVTGAKPGEWVAIYGIGGLGHVAVQYAKAMGLNVVAVDIGDEKLELAKELGADLVVNPLKEDAAKFMKEKVGGVHAAVVTAVSKPAFQSAYNSIRRGGACVLVGLPPEEMPIPIFDTVLNGIKIIGSIVGTRKDLQEALQFAAEGKVKTIIEVQPLEKINEVFDRMLKGQINGRVVLTLEDK

(19) United States
(12) Patent Application Publication
(10) Pub. No.: US 2025/0019732 A1
(43) Pub. Date: Jan. 16, 2025

(54) METHOD FOR CHEMICAL-BIOLOGICAL CASCADE SYNTHESIS OF L-PHOSPHINOTHRICIN AND MUTANTS THEREFOR

(71) Applicant: ZHEJIANG UNIVERSITY OF TECHNOLOGY, Hangzhou (CN)

(72) Inventors: Yaping XUE, Hangzhou (CN); Feng Cheng, Hangzhou (CN); Shuping Zou, Hangzhou (CN); Jianmiao XU, Hangzhou (CN); Yuguo ZHENG, Hangzhou (CN)

(21) Appl. No.: 18/764,955

(22) Filed: Jul. 5, 2024

(30) Foreign Application Priority Data
Jul. 7, 2023 (CN) 202310827168.9
Jun. 24, 2024 (CN) 202410819732.7

(51) Int. Cl. C12P 9/00; C12N 1/20; C12N 9/04

(52) U.S. Cl. CPC C12P 9/00; C12N 1/205; C12N 9/0006; C12N 2510/00; C12Y 101/01001

(57) ABSTRACT

A method for chemical-biological cascade synthesis of L-phosphinothricin is carried out as follows: 3-(methylethoxyphosphinyl) ethyl propionate is synthesized by addition reaction from diethoxymethylphosphine and acrylic acid, then a condensation reaction is carried out with 3-(methylethoxyphosphinyl) ethyl propionate and sodium ethoxide as reactants, then the product is subjected to a hydrolysis reaction with diethyl oxalate to synthesize 4-(hydroxymethylphosphinyl)-2-oxobutyric acid, and finally, L-phosphinothricin is catalytically synthesized by taking 4-(hydroxymethylphosphinyl)-2-oxobutyric acid as a raw material, and using highly active and stable wet cells co-expressing phsophinothricin dehydrogenase and alcohol dehydrogenase or co-expressing a phsophinothricin dehydrogenase mutant and alcohol dehydrogenase as a biocatalyst, thereby solving the problems of existing L-phosphinothricin synthesis being tedious, low asymmetric amination reduction activity and poor stability.

### REFERENCE TO AN ELECTRONIC SEQUENCE LISTING

The content of the electronic sequence listing (2024-07-05-Sequence.xml; Size: 7 KB; and Date of Creation: Jun. 28, 2024) is herein incorporated by reference in its entirety.

### TECHNICAL FIELD

The present invention belongs to the technical field of bioengineering, chemistry and chemical engineering, and specifically relates to biocatalysts, mutants, a method for chemical-biological cascade synthesis of L-phosphinothricin.

### BACKGROUND TECHNOLOGY

Phosphinothricin (PPT), whose chemical name is 2-amino-4-(hydroxy (methyl) phosphinyl)-butyric acid, has the characteristics of high herbicidal activity, wide herbicidal spectrum and good environmental compatibility, and is very suitable for developing herbicide resistance genes. Phosphinothricin resistance genes have been introduced into various crops such as rice, wheat, corn, beet, tobacco, soybean, cotton, potato, tomato, rape, sugarcane, etc. In recent years, these transgenic crops have been popularized and planted in agricultural large countries such as American, Asian, Europe, Australian and the like, and have a very broad application prospect. However, phosphinothricin sold on the market is generally a racemic mixture. Wherein, 50% of D-phosphinothricin has no herbicidal activity, only L-configuration has phytotoxicity which is easy to decompose in soil, has low toxicity to humans and animals, and has a small environmental pollution. If the phosphinothricin product can be used in the form of an optical pure isomer of L-configuration, the amount of phosphinothricin can be reduced by 50%, which is of great significance for improving the atom economy, reducing the cost and reducing the environmental pressure.

The methods for generating L-phosphinothricin include chemical synthesis, fermentation, and biosynthesis. The enzymes involved in the biosynthesis method mainly include protease, deacetylase, transaminase, amidase, ester hydrolase and nitrile hydratase. In a plurality of enzymatic synthesis routes of generating phosphinothricin, a ketone carbonyl group of the keto acid intermediates is a latent chiral functional group that can be used to constructchiral centers through enzymatic synthesis approaches, and the keto acid route has also become a suitable route for the industrial development and production of L-phosphinothricin due to its low cost and easy availability of raw materials, as well as the ability to avoid the use of highly toxic cyanide.

The biocatalytic method for producing phosphinothricin has the advantages of strict stereoselectivity, mild reaction conditions, and high yield, making it an advantageous method for producing L-phosphinothricin. It mainly includes the following three categories:
1) an L-phosphinothricin derivative is used as a substrate, L-phosphinothricin is obtained by means of enzymatic direct hydrolysis, and the main advantages are high conversion rate and high product e.e. value, but expensive and difficult-to-obtain chiral raw materials are required as precursors, so that the cost is increased, which is not conducive to industrial production. For example, the simplest method for preparing L-phosphinothricin by a biological method is to directly hydrolyze bialaphos by using protease. Bialaphos is a natural tripeptide compound, and bialaphos loses 2 molecules of L-alanine under the catalysis of protease to generate L-phosphinothricin.
2) The precursor of racemic phosphinothricin is used as a substrate, and L-phosphinothricin is obtained by selective resolution under the action of an enzyme. The main advantages of the method are that the raw materials are relatively easy to obtain, and the activity of the catalyst is high, but the theoretical yield of the phosphinothricin can only reach 50%, which can cause waste of the raw materials. For example, Cao et al. (Cao C-H, Cheng F, Xuc Y-P, Zheng Y-G (2020) Efficient synthesis of L-phosphinothricin using a novel aminoacylase mined from Stenotrophomonas maltophilia. Enzyme and Microbial Technology 135 doi: 10.1016/j.enzmictec.2019.109493) conduct a chiral resolution with a novel aminoacylase mined from Stenotrophomonas maltophilia using N-acetyl-PPT as a substrate to obatin L-phosphinothricin. Optically pure L-PPT (>99.9% e.e.) is acquired with high conversion (>49%) within 4 h under the catalysis of the whole cells.
3) Taking 4-(hydroxymethylphosphinyl)-2-oxobutyric acid (PPO) as a substrate, L-phosphinothricin is obtained by asymmetric synthesis under the action of an enzyme, and the mainly involved enzyme comprises transaminase and phosphinothricin dehydrogenase. Bartsch (Bartsch K (2005) Process for the Preparation of L-Phosphinthrcine by Enzymatic Transaction with Aspartate. U.S. Pat. No. 6,936,444 B1) uses PPO as a substrate and L-aspartic acid as an amino donor, conducts enzymatic chiral synthesis of L-phosphinothricin with the transaminase which has specific enzyme activity for PPO and L-aspartic acid and is screened and separated from soil microorganisms, and when the substrate concentration is 552 mM, the conversion rate still reaches 52% after reacting at very high temperature (80° C.) for 4 hours, and the space-time yield is 4.5 g L-PPT/g·L⁻¹·d⁻¹. However, there are two major drawbacks to using transaminase to prepare L-phosphinothricin. Firstly, this is a reversible reaction, and the raw material PPO cannot be completely converted to L-PPT, and the conversion rate cannot reach 100%; secondly, in order to make the reversible reaction proceed in the direction of generating L-PPT, at least twice the amount of L-aspartic acid as an amino donor is required, and the excessive amount of aspartic acid brings a significant challenge for the separation of L-PPT.

The phosphinothricin dehydrogenase has two types: NADH-dependent type and NADPH-dependent type, and both can utilize an inorganic ammonia donor to perform an asymmetric amination reaction with PPO as a substrate, so as to synthesize L-phosphinothricin. The difference is that different types of phosphinothricin dehydrogenase require different coenzyme: when NADH-dependent phosphinothricin dehydrogenase is subjected to the catalytic reaction, coenzyme NADH (CN114350631A) is required, while NADPH-type phosphinothricin dehydrogenase needs NADPH (CN 109609475A). The conversion rate can reach 100%. However, the chemical synthesis of substrate PPO is also crucial for the preparation of L-phosphinothricin, and a clean and safe synthetic process is the key to success. The traditional synthesis process of D, L-phosphinothricin involves highly toxic cyanide, so searching a similar synthesis process for PPO from the traditional route does not conform to the concept of environmental protection.

To this end, according to the present invention, PPO is synthesized from diethoxymethylphosphine through addition, condensation and hydrolysis reactions, the use of highly toxic cyanide is eliminated, and the amount of three wastes produced is reduced by more than 50% compared with the method using D,L-phosphinothricin. Further, L-phosphinothricin is asymmetrically synthesized by a biological inorganic amination reaction. The whole chemical-enzymatic synthesis process is simple, the reaction conditions are mild, and industrial amplification is easy to realize continuous production.

### SUMMARY OF THE INVENTION

The objective of the present invention is to provide a method for synthesizing L-phosphinothricin by chemical-biological cascade synthesis and a mutant therefor. The present invention is carried out as follows: 3-(methylethoxyphosphinyl) ethyl propionate is synthesized by addition reaction from diethoxymethylphosphine and acrylic acid, then a condensation reaction is carried out with 3-(methylethoxyphosphinyl) ethyl propionate and sodium ethoxide as reactants, then the product is subjected to a hydrolysis reaction with diethyl oxalate to synthesize 4-(hydroxymethylphosphinyl)-2-oxobutyric acid, and finally, L-phosphinothricin is catalytically synthesized by taking 4-(hydroxymethylphosphinyl)-2-oxobutyric acid as a raw material, and using highly active and stable wet cells co-expressing phsophinothricin dehydrogenase and alcohol dehydrogenase or co-expressing a phsophinothricin dehydrogenase mutant and alcohol dehydrogenase as a biocatalyst, thereby solving the problems of existing L-phosphinothricin synthesis being tedious, low asymmetric amination reduction activity and poor stability.

The technical solutions adopted by the present invention are as follows:

A method for synthesis of L-phosphinothricin, and the method comprises the following steps:

forming a reaction system by using the wet cells obtained by fermentative cultivation of an engineered strain of recombinant Escherichia coli that co-express the gene encoding phosphinothricin dehydrogenase and the gene encoding alcohol dehydrogenase, or that co-express the gene encoding a phosphinothricin dehydrogenase mutant and the gene encoding alcohol dehydrogenase as a catalyst, using 4-(hydroxymethylphosphinyl)-2-oxobutyric acid as a substrate, adding isopropanol and NAD+, and using a buffer solution with a pH of 6-8 as the reaction medium, conducting the conversion reaction at 40-60° C. and 100-200 rpm, and subjecting the reaction solution to separation and purification to obtain L-phosphinothricin; wherein the phosphinothricin dehydrogenase mutant is obtained by single or double mutation at position 73 or 91 of the phosphinothricin dehydrogenase with the amino acid sequence shown as SEQ ID NO.2.

Preferably, the preparation of 4-(hydroxymethylphosphinyl)-2-oxobutyric acid comprises:
(1) synthesizing 3-(methylethoxyphosphinyl) ethyl propionate by addition reaction from diethoxymethylphosphine and acrylic acid (preferably 98% of purity) under the conditions of temperature 100-150° C. and pressure 0.5-5 MPa;
(2) conducting the condensation reaction of 3-(methylethoxyphosphinyl) ethyl propionate with sodium ethoxide and diethyl oxalate under the conditions of temperature 40-80° C. and pressure 1-5 MPa, then adjusting the pH value of the reaction solution to 6-8 (preferably 7.5), subjecting the reaction solution to heating and hydrolysis reaction at 50-70° C., rectifying the reaction liquid to recover the byproduct ethanol, then filtering to remove sodium chloride, thereby obtaining 4-(hydroxymethylphosphinyl)-2-oxobutyric acid.

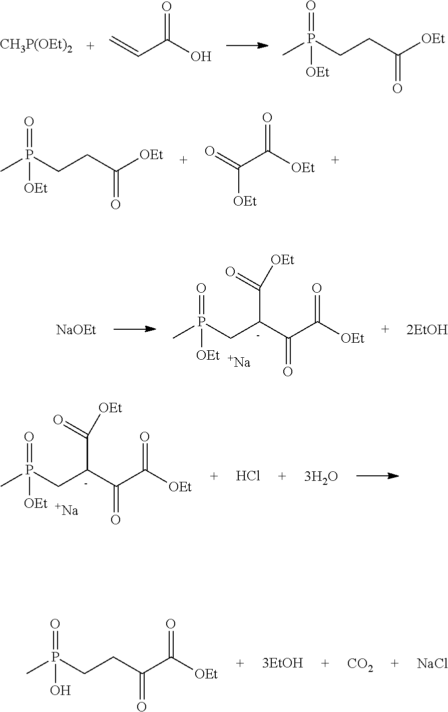

Preferably, the reaction in step (1) is performed for 6 h at a temperature of 120° C. and a pressure of 1 MPa. The volume ratio of acrylic acid to diethoxymethylphosphine is 1-5:1, preferably 1.1:1.

Preferably, in step (2), the condensation reaction is performed for 8 h at a temperature of 60° C. and a pressure of 1.5 MPa; and the temperature of the hydrolysis reaction is 60° C. The volume of 3-(methylethoxyphosphinyl) ethyl propionate is 20-50 L/kg, preferably 36.5 L/kg in terms of the mass of sodium ethylate; and the mass ratio of sodium ethylate to diethyl oxalate is 1:1-3, preferably 1:1.

Preferably, in the reaction system, the final concentration of the wet cells added to the reaction system is 10-40 g/L (more preferably 10 g/L), and the final concentration of the substrate added to the reaction system is 200-800 mM, more preferably 200 mL; the final concentration of isopropanol added to the reaction system is 200-1000 mM, more preferably 300 mL; and the final concentration of NAD+added to the reaction system is 0.4-0.8 mM, more preferably 0.6 mM; and the reaction medium is preferably a phosphate buffer solution having a pH of 7.5.

Preferably, the phsophinothricin dehydrogenase mutant is obtained by mutating the amino acid sequence shown as SEQ ID NO. 2 in one of the following ways: (1) mutating valine at position 73 into cysteine, V73C; (2) mutating methionine at position 91 into glycine, M91G; or (3) mutating valine at position 73 into cysteine and methionine at position 91 into glycine, V73C-M91G.

The amino acid sequence of the phosphinothricin dehydrogenase is shown as SEQ ID NO. 2, and the nucleotide sequence of the coding gene is shown as SEQ ID NO. 1; and the amino acid sequence of the alcohol dehydrogenase is shown as SEQ ID NO. 4, and the nucleotide sequence of the coding gene is shown as SEQ ID NO. 3.

Preferably, the preparation method of the catalyst comprises: inoculating the engineered strain of recombinant Escherichia coli co-expressing the gene encoding phsophinothricin dehydrogenase and the gene encoding alcohol dehydrogenase, or co-expressing the gene encoding the phsophinothricin dehydrogenase mutant and the gene encoding alcohol dehydrogenase into an LB liquid culture medium containing ampicillin with a final concentration of 50 μg/ml, culturing it at 37° C. for 8 hours, then inoculating it into a fresh LB liquid culture medium containing ampicillin with a final concentration of 50 μg/ml, culturing it at 37° C. and 180 r/min for 2 hours, then adding IPTG with a final concentration of 0.1 mM into the culture solution, culturing it at 18° C. for 14 hours, and centrifuging it at 4° C. and 8000 rpm for 10 minutes to obtain corresponding wet cells. The wet cells produce a corresponding protein, which can be used for preparing a pure enzyme solution or preparing immobilized cells.

Preferably, the engineered strain of recombinant Escherichia coli that co-express the gene encoding phsophinothricin dehydrogenase and the gene encoding alcohol dehydrogenase is constructed according to the following method: the alcohol dehydrogenase gene ADH (nucleotide sequence shown in SEQ ID No.3) is constructed into the recombinant expression vector pETDuet-LcGluDH at the NdeI and AvrIIsites of the multiple cloning site 2 (MCS2) through One Step Cloning Kit of Vazyme to obtain a co-expression vector pETDuet-LcGluDH-ADH, which is then transformed into E. coli BL21(DE3) to obtain the co-expression strain of phsophinothricin dehydrogenase and alcohol dehydrogenase, i.e., E. coli BL21(DE3)/pETDuet-LcGluDH-ADH. Using the same method, the engineered strain of recombinant Escherichia coli that co-express the gene encoding the phsophinothricin dehydrogenase mutant and the gene encoding alcohol dehydrogenase is constructed.

The invention also provides a phosphinothricin dehydrogenase mutant for synthesizing L-phosphinothricin, wherein the phosphinothricin dehydrogenase mutant is obtained by performing single mutation or double mutation at position 73 or 91 of the phosphinothricin dehydrogenase shown in SEQ ID NO. 2, and preferably the amino acid sequence shown in SEQ ID NO. 2 is mutated in one of the following ways: (1) mutating valine at position 73 into cysteine, V73C; (2) mutating methionine at position 91 into glycine, M91G; or (3) mutating valine at position 73 into cysteine and methionine at position 91 into glycine, V73C-M91G.

Compared with the prior art, the beneficial effects of the present invention are mainly embodied:
1) The traditional route obtains L-phosphinothricin from existing D,L-phosphinothricin by resolution, and the route of the present invention innovatively adopts a chemical-biological cascade synthesis technology, and uses conventional compounds to replace D,L-phosphinothricin as raw materials to produce L-phosphinothricin, which abandons the technical route of firstly synthesizing D,L-phosphinothricin via a Strecker reaction and then subjecting D,L-phosphinothricin to resolution to obtain L-phosphinothricin.
2) The process route of the present invention is environmental friendly, since D,L-phosphinothricin is not required to be synthesized in the route, the use of highly toxic cyanide needed for synthesizing D,L-phosphinothricin is avoided, the amount of three wastes is reduced by more than 50% compared with the route which uses D,L-phosphinothricin.
3) The process of the present invention is simple, chemical reaction steps and biological reaction steps have mild conditions, and industrial amplification is easy to realize continuous production.
4) By coupling the phosphinothricin dehydrogenase mutant of the present invention and the alcohol dehydrogenase, the conversion rate of the biocatalytic step reaches 100%, no substrate residue exists, the ee value of the product is greater than 99%, and compared with the existing method, separation and purification are convenient.

### BRIEF DESCRIPTION OF THE DRAWINGS

FIG. 1 is a schematic diagram of a reaction equation for biosynthesis of L-phosphinothricin.

FIG. 2 is a physical graph of the pETDuet-Cluxurium-ADH recombinant plasmid in Example 3.

FIG. 3 is an SDS-PAGE diagram in Example 3, wherein lane 1 is a protein molecular weight Marker, and lane 2 is the supernatant obtained by ultrasonic disruption of wet cells co-expressing the phosphinothricin dehydrogenase LcGluDH and the alcohol dehydrogenase ADH.

FIG. 4 is an HPLC spectral analysis diagram of a 4-(hydroxymethylphosphinyl)-2-oxobutyric acid standard.

FIG. 5 is an HPLC spectrum analysis diagram of a D-PPT standard and an L-PPT standard.

FIG. 6 is a diagram of the reaction process using recombinant E. coli BL 21 (DE3)/psidera UET-C Luxu-ADH and a mutant to catalyze 200 mM 4-(hydroxymethylphosphinyl)-2-oxobutyric acid.

FIG. 7 is a diagram of the reaction process using recombinant E. coli BL 21 (DE3)/Psiwu UET-Copera-ADH and a mutant mM to catalyze 800 4-(hydroxymethylphosphinyl)-2-oxobutyric acid.

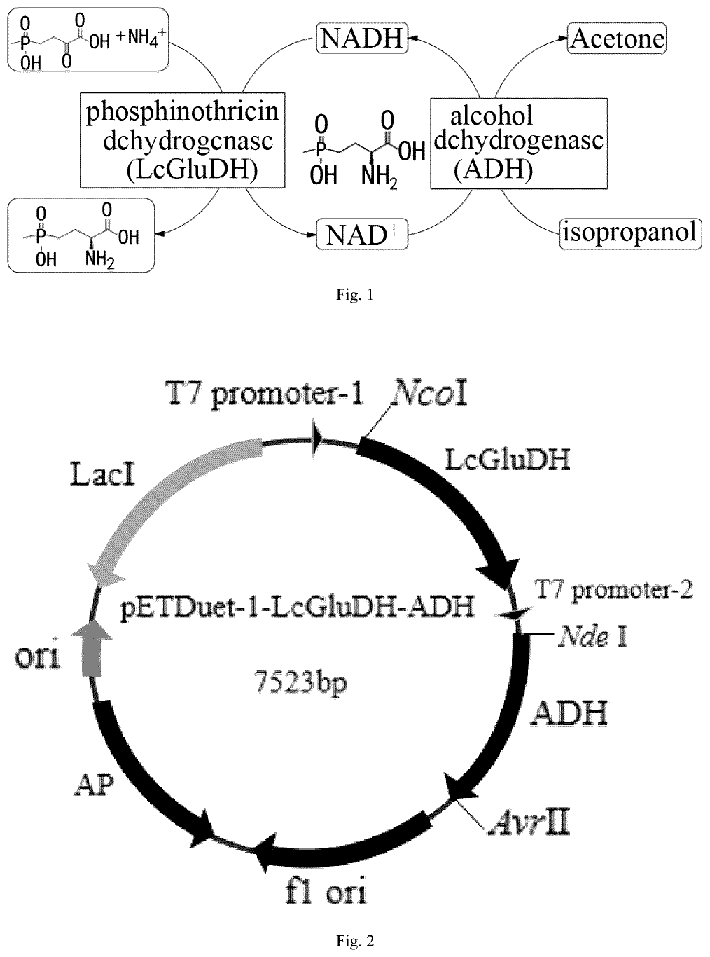

### DETAILED DESCRIPTION OF THE INVENTION

The present disclosure will be further described below with reference to specific examples, but the scope of protection of the present disclosure is not limited thereto:

### Example 1: Large-Scale Chemical Synthesis of 3-(Methylethoxyphosphinyl) Ethyl Propionate

2000 L of diethoxymethylphosphine was injected in a 5 m³ high-pressure reactor. Under stirring at 100 rpm, 2200 L of acrylic acid (content: 98%) was added for addition reaction. The reaction temperature was controlled at 120° C. and the pressure at 1 MPa. After 6 hours of reaction, the reaction mixture was processed through a small distillation column to recover the by-product ethanol, and then filtered to obtain 2500 L of 3-(methylethoxyphosphinyl) ethyl propionate. The yield of the addition reaction was 96% by HPLC.

### Example 2: 4-(Hydroxymethylphosphinyl)-2-Oxobutyric Acid is Synthesized from 3-(Methylethoxyphosphinyl) Ethyl Propionate

3000 L of 3-(methylethoxyphosphinyl) ethyl propionate prepared by the method of Example 1 and 82 kg of sodium ethoxide were added to a 5 m³ condensation reactor, 86 kg of diethyl oxalate was added, the reaction was carried out at a reaction temperature of 60° C. and a pressure of 1.5 MPa for 8 hours; then quantitative hydrochloric acid (measured by reaction pH) was added to adjust pH value to 7.5; and then a heating reaction was performed by controlling the reaction temperature to be 60° C., and meanwhile the byproduct ethanol was evaporated for rectification to recover 95% ethanol. After the reaction was completed, sodium chloride was removed through filtration to obtain a 4-(hydroxymethylphosphinyl)-2-oxobutyric acid solution, the concentration of the solution was 10 mol/L, the volume was 3.2 L, and the pH value was measured to be 1.5.

### Example 3: Construction and Expression of Phosphinothricin Dehydrogenase Parent Engineered Strain and Starting Co-Expression Strain

1. Phosphinothricin Dehydrogenase Parent Engineered Strain E. coli BL21(DE3)/pETDuet-LcGluDH

The amino acid sequence of the LcGluDH gene (NCBI accession number was 8183) from Lysinibacillus composti was codon optimized (the codon optimized nucleotide sequence is shown in SEQ ID No.1, and the amino acid sequence is shown in SEQ ID No.2), gene synthesis was performed by Hangzhou Qinke Biotechnology Co., Ltd. to obtain the LcGluDH gene, the LcGluDH gene was cloned into plasmid pETDuet at the Ncol site of MCS1 (multiple cloning site 1) to construct the recombinant expression vector pETDuet-LcGluDH, the His-Tag gene of the plasmid itself was retained, and the recombinant expression vector pETDuet-LcGluDH was transformed into E. coli BL21(DE 3) and sent to Hangzhou Qinke Biotechnology Co., Ltd. for the production of phosphinothricin dehydrogenase parent engineered strain E. coli BL21(DE3)/pETDuet-LcGluDH.

The phosphinothricin dehydrogenase parent engineered strain E. coli BL21(DE3)/pETDuet-LcGluDH was inoculated into an LB liquid culture medium containing ampicillin with a final concentration of 50 μg/ml, cultured at 37° C. for 8 hours, inoculated into a fresh LB liquid culture medium containing 50 μg/ml ampicillin at an inoculation volume of 2%, cultured at 37° C. and 180 rpm for 2 hours, then IPTG was added to the culture at a final concentration of 0.1 mM, the resulting culture was cultured at 18° C. for 14 hours and then centrifuged at 4° C. for 10 minutes, thereby obtaining wet cells of the phosphinothricin dehydrogenase parent engineered strain.

2. The Starting Co-Expression Strain E. coli BL21(DE3)/pETDuet-LcGluDH-ADH

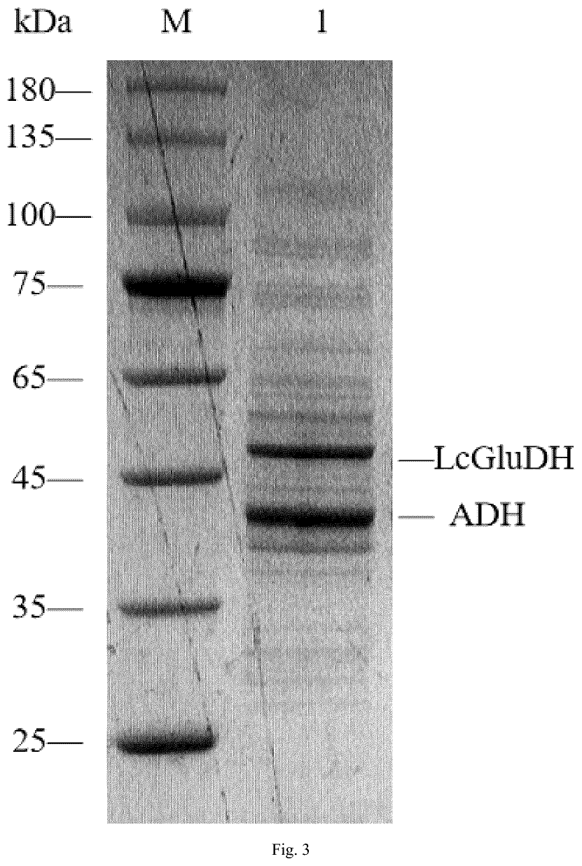

Then, the alcohol dehydrogenase gene ADH (the PDB accession number was 1 rjw, the nucleotide sequence was shown in SEQ ID No.3 and the amino acid sequence was shown in SEQ ID No.4), which was cloned from Bacillus stearothermophilus, was constructed into the recombinant expression vector pETDuet-LcGluDH at the NdeI and AvrIIsites of the multiple cloning site 2 (MCS2) through One Step Cloning Kit of Vazyme to obtain a co-expression vector pETDuet-LcGluDH-ADH (plasmid map shown in FIG. 2), and the co-expression vector pETDuet-LcGluDH-ADH was then transformed into E. coli BL21(DE3), thereby obtaining the starting co-expression strain of phosphinothricin dehydrogenase parent and alcohol dehydrogenase E. coli BL21(DE3)/pETDuet-LcGluDH-ADH.

The starting co-expression strain E. coli BL21(DE3)/pETDuet-LcGluDH-ADH was inoculated into an LB liquid medium containing ampicillin at a final concentration of 50 μg/mL, cultured at 37° C. for 8 hours, then inoculated into a fresh LB liquid medium containing 50 μg/mL ampicillin at an inoculation volume of 2%, and cultured at 37° C. and 180 rpm for 2 hours, then IPTG was added to the culture at a final concentration of 0.1 mM, the resulting culture was cultured at 18° C. for 14 hours and then centrifuged for 10 min at 4° C. and 8000 rpm, thereby obtaining wet cells of the starting co-expression strain.

0.2 g of wet cells were taken and suspended with 10 mL of binding buffer (pH 7.4, 100 mM sodium phosphate buffer containing 0.3 M NaCl), the resulting suspension was ultrasonically disrupted for 15 minutes (ice bath, power of 400 W, disrupting for 1 second, and pausing for 5 seconds) and centrifuged at 4° C. and 12000 rpm for 20 min, the resulting supernatant was taken and subjected to agarose gel electrophoresis, and the SDS-PAGE diagram was shown in FIG. 3.

### Example 4: Construction and Screening of Phosphinothricin Dehydrogenase Mutant Co-Expression Library

### 1. Phosphinothricin Dehydrogenase Mutant Co-Expression Library

The preparation of the phosphinothricin dehydrogenase mutant co-expression library was achieved through random saturation mutagenesis. Primers were designed according to Table 1. Using the vector pETDuet-LcGluDH-ADH as a template, site-directed saturation mutagenesis PCR was conducted. The PCR products were subjected to agarose gel electrophoresis for positive validation. After digestion with DpnI enzyme at 37° C. and 220 r/min for 1 hour, followed by inactivation at 65° C. for 1 minute, the PCR products were heat-shocked for transformation. E. coli BL21(DE3) was activated and cultured at 37° C. and 220 rpm for 1 hour. Subsequently, the transformed cells were spread on LB plate containing 50 μg/mL ampicillin resistance and incubated overnight at 37° C. The colonies that grew out were then picked, constituting the phosphinothricin dehydrogenase mutant co-expression library.

The mutagenesis PCR system (100 μL): 25 μL of 2×DNA Taq polymerase buffer, 1 μL of dNTPs, 1 μL each of the upper and lower mutagenic primers, 1 μL of template, 0.5 μL of DNA Taq polymerase, 1 μL of 10 mM MnCl₂ solution, and a complement of ddH2O to 50 μL.

PCR conditions: pre-denaturation at 95° C. for 5 minutes, 30 cycles: 90° C. for 30 seconds, 60° C. for 30 seconds, 72° C. for 2 minutes, and final extension at 72° C. for 5 minutes.

TABLE 1
Design of Phosphinothricin Dehydrogenase Site-
Specific Saturation Mutation

| PRIMER | |
|---|---|
| NAME | Primer sequence (5′-3′) |
| LcGluDH-73-F | AGGTGGCNNKCGTTTTCATCCTATGGTTTCTGAA |
| LcGluDH-73-R | GAAAACGMNNGCCACCTTTGGTGGGACCTACT |
| LcGluDH-91-F | CATGTGGNNKACCCTGAAGTGCGGGATTGTAG |
| LcGluDH-91-R | TCAGGGTMNNCCACATGCTCAGTGCTTTAACTTC |

Note:
N in Table 1 represents A/C G/T, M represents A/C, and K represents G/T.

### 2. Inducible Expression of Phosphinothricin Dehydrogenase Mutant Co-Expressing Engineered Strain

The strain in step 1 was inoculated into an LB liquid culture medium containing ampicamycin with a final concentration of 50 μg/ml, cultured at 37° C. for 8 hours, inoculated at an inoculation volume of 2% into a fresh LB liquid culture medium containing ampicamycin with a final concentration of 50 μg/ml, cultured for 2 hours at 37° C. and 180 rpm, then IPTG was added to the culture solution at a final concentration of 0.1 mM, and after being cultured at 18° C. for 14 hours, centrifugation was performed at 4° C. and 8000 rpm for 10 minutes to obtain corresponding wet cells.

The obtained cells produce corresponding proteins, which can be used for preparing pure enzyme liquid, and can also be used for preparing immobilized cells.

### 3. Screening of Co-Expression Library of the Mutant

1 mL of a reaction system was prepared by using the starting co-expression wet cells of phosphinothricin dehydrogenase and alcohol dehydrogenase prepared by the method of example 3, or the co-expression wet cells of the phosphinothricin dehydrogenase mutant and alcohol dehydrogenase prepared by the method of example 2 as the catalyst, the intermediate product 4-(hydroxymethylphosphinyl)-2-oxobutyric acid as the substrate, isopropanol as the auxiliary substrate, trace NAD⁺ as the exogenous source, and a PH 7.5, 100 mM phosphate buffer served as the reaction medium, in which, the amount of the catalyst calculated based on the final concentration of the wet cells was 10 g/L, the final concentration of the substrate is 100 mM, the final concentration of isopropanol is 120 mM, and the final concentration of NAD⁺ was 0.4 mM. The reaction system was subjected to reaction at 55° C. and 600 rpm for 5 min, then 50 μL of the resulting reaction solution was taken, 5 μL of hydrochloric acid was added to terminate the reaction, the reaction solution was diluted by 100 times with ultrapure water, 200 μL of the diluted reaction solution and 400 μL of derivative reagent (pH9.8 boronic acid buffer containing 15 mM o-phthalaldehyde and 15 mM N-acetyl-L-cysteine) were taken and subjected to derivatization at 30° C. for 5 min, 400 μL of ultrapure water was added to make up to 1 mL, the resulting solution was centrifuged at 12000 rpm for 1 minute, the supernatant was collected and passed through a 0.22 μM microfilter, the filtrate was collected as the liquid phase sample, and 4-(hydroxymethylphosphinyl)-2-oxobutyric acid, L-phosphinothricin, D-phosphinothricin and e.e. values were detected by liquid phase detection. Using the product L-phosphinothricin and e.e. values as indicators, screening for advantageous mutants was conducted, and experimental results were shown in tables 2 and 3.

Liquid phase detection conditions of 4-(hydroxymethylphosphinyl)-2-oxobutyric acid were as follows: chromatographic column Unary® 18 (4.6×250 mm, ACCHROM, China), mobile phase was acetonitrile: 50 mM ammonium dihydrogen phosphate solution (PH 3.8, containing 10% tetrabutylammonium hydroxide) with a volume ratio of 12:88, the flow rate was 1 mL/min, detection wavelength was 232 nm, sample injection amount was 10 μL, column temperature was 40° C., and retention time of 4-(hydroxymethylphosphinyl)-2-oxobutyric acid was 11.1 minutes.

Liquid phase detection conditions of phosphinothricin were as follows: chromatographic column Unary® 18 (4.6×250 mm, ACCHROM, China), mobile phase was methanol: 0.05 M ammonium acetate (pH 5.7) with a volume ratio of 10:90, flow rate was 1.0 mL/min, detection wavelength Ex=340 nm, Em=450 nm, sample injection amount was 10 μL, and column temperature was 35° C. The retention time of L-phosphinothricin and D-phosphinothricin was 13 minutes and 15.5 minutes, respectively.

TABLE 2
Conversion Rate of Multienzyme Catalytic Reaction after Site-
Specific Saturation Mutagenesis of LcGluDH (5 min Reaction)

| Mutation at | conversion | Mutation at | conversion |
|---|---|---|---|
| position 73 | (%) | position 91 | (%) |
| WT | 12 | WT | 12 |
| V73A | N.D. | M91A | N.D. |
| V73N | N.D. | N.D. | N.D. |
| V73D | N.D. | M91D | N.D. |
| V73C | 45 | M91C | 12 |
| V73Q | N.D. | M91Q | 3.4 |
| V73E | 3.2 | M91E | N.D. |
| V73G | 10.1 | M91G | 60 |
| V73H | N.D. | M91H | 10.8 |
| V73I | 10.4 | M91I | N.D. |
| V73L | N.D. | M91L | N.D. |
| V73K | N.D. | M91K | 2.2 |
| V73M | 12 | M91M | 2.6 |
| V73F | N.D. | M91F | N.D. |
| V73P | 4.5 | M91P | N.D. |
| V73S | N.D. | M91S | N.D. |
| V73T | N.D. | M91T | N.D. |
| V73W | N.D. | M91W | N.D. |
| V73Y | N.D. | M91Y | 2.1 |
| V73R | N.D. | M91V | N.D. |

N.D.: Not Detected

TABLE 3
Catalytic performance and stereoselectivity of LcGluDH
and mutants thereof (co-expression strains)

| | L-phosphinothricin | conversion | e.e |
|---|---|---|---|
| Mutation Site | (mM) | (%) | (%) |
| LcGluDH-ADH | 1.482 | 12 | 99.9 |
| LcGluDH-V73C-ADH | 20.265 | 45 | 99.9 |
| LcGluDH-M91G-ADH | 26.525 | 60 | 99.9 |
| LcGluDH-V73C-M91G-ADH | 45.432 | 80.1 | 99.9 |

Note:
the conversion rate was obtained under the conditions of 100M substrate and reaction for 5 min.

Table 2 showed that the strain V73C with improved vitality was screened by V73 site-saturation mutagenesis and screening; and the strain M91G with improved vitality was screened by M91 site-saturation mutagenesis and screening.

It could be seen from Table 3 that under the condition of adding exogenous NAD⁺, phosphinothricin dehydrogenase mutant V73C could increase the conversion rate of 100 mM PPO from 12% to 45%. Under the condition of adding exogenous NAD⁺, the mutant of phosphinothricin dehydrogenase M91G could increase the conversion rate of 300 mM substrate from 12% to 60%. Then two mutation sites of V73C and M91G were subjected to combinatorial mutation, it was found that the activity was further improved, and the conversion rate of 100 M substrate reached 80.1%.

### Example 5: Purification of Phosphinothricin Dehydrogenase Parent Strain and Mutants Thereof

According to the results of catalytic performance of co-expression engineered strains of the phosphinothricin dehydrogenase mutants screened in Example 4, the wet cells of phosphinothricin dehydrogenase parent engineered E. coli strain BL21(DE3)/pETDuet-LcGluDH prepared in Example 3 were used to construct engineered strains expressing phosphinothricin dehydrogenase alone (E. coli BL21(DE3)/pETDuet-LcGluDH-V73C, E. coli BL21(DE3)/pETDuet-LcGluDH-M91G, E. coli BL21(DE3)/pETDuet-LcGluDH-V73C-M91G). Wet cells of the phosphinothricin dehydrogenase mutants were prepared according to the method for preparing wet cells of the parent engineered strain in Example 3.

Each 0.2 g of wet cells of engineered strains of phosphinothricin dehydrogenase parent and phosphinothricin dehydrogenase mutants were respectively suspended with 10 mL of binding buffer (pH 7.4, 100 mM sodium phosphate buffer containing 0.3 M NaCl), the resulting suspension was ultrasonically disrupted for 15 minutes (ice bath, power 400W, disrupting for 1 second, pausing for 5 seconds) and centrifuged at 4° C. and 12000 rpm for 20 min, and supernatant was taken as a sample.

The protein purification process using a Ni affinity column (1.6×10 cm, Bio-Rad, USA) was carried out as follows:
1. the Ni column was equilibrated with 5 column volumes of binding buffer (pH 7.4, 50 mM sodium phosphate buffer comprising 0.3M NaCl) until the baseline stabilized;
2. the sample was loaded onto the column at a flow rate of 1 mL/min, the sample volume was adjusted to contain 200 mg of protein, allowing the target protein to bind to the Ni column;
3. impurities were washed away with 6 column volumes of buffer A (pH 7.4, 50 mM sodium phosphate buffer comprising 0.3M NaCl and 30 mM imidazole) at a flow rate of 1 mL/min until the baseline stabilized;
4. the target protein was eluted with buffer B (pH 7.4, 50 mM sodium phosphate buffer comprising 0.3M NaCl and 500 mM imidazole) at a flow rate of 1 mL/min, 6 column volumes were eluted and the eluted protein was collected; the collected protein was dialyzed overnight against 20 mM phosphate buffer (pH 7.4) using a dialysis bag with a molecular weight cutoff of 10 kDa; the dialyzed solution was collected to obtain 10 mL of pure enzyme solution of phosphinothricin dehydrogenase and 10 mL of pure enzyme solution of the phosphinothricin dehydrogenase mutant;
5. the Ni column was washed with 5 column volumes of binding buffer (pH 8.0, 50 mM sodium phosphate buffer comprising 0.3M NaCl) until the baseline stabilized, and stored with a 5×-column volume of ultrapure water containing 20% ethanol.

The protein concentration of the pure enzyme solutions were determined by using a BCA protein determination kit (Nanjing Kaibased Biotechnology Development Co., Ltd., Nanjing), and the results were shown in Table 4.

TABLE 4
PROTEIN CONCENTRATION OF PURE ENZYME
SOLUTIONS OF PHOSPHINOTHRICIN DEHYDROGENASE
PARENT AND MUTANTS

| Enzyme | protein concentration | |
|---|---|---|
| LcGluDH | 0.24 | (μg/μL) |
| LcGluDH-V73C | 0.5 | (μg/μL) |
| LcGluDH-M91G | 0.4 | (μg/μL) |
| LcGluDH-V73C-M91G | 0.22 | (μg/μL) |

### Example 6: Specific Enzyme Activity Assay of Phosphinothricin Dehydrogenase Parent and Mutants Thereof

The enzyme activity unit (U) is defined as follows: under the conditions of 55° C. and PH 7.5, the amount of enzyme required to generate 1 μmol of L-phosphinothricin per minute was defined as an enzyme activity unit, U. The specific enzyme activity was defined as the number of activity units per milligram of enzyme, U/mg.

The standard conditions for enzyme activity detection were as follows: the pure enzyme of phosphinothricin dehydrogenase parent or its mutants with a protein content of 0.22 mg prepared by the method of Example 5 was taken and added with 4-(hydroxymethylphosphinyl)-2-oxobutyric acid at a final concentration of 100 mM and coenzyme NADH at a final concentration of 10 mM, thereby forming a reaction system, whose total volume was 10 mL; the reaction was carried out for 10 minutes under the conditions of 55° C., pH 7.5 and 600 rpm, HPLC detection and analysis were carried out by adopting the method in Example 4, and the results were shown in Table 5.

TABLE 5
Specific Enzyme Activity of phosphinothricin
dehydrogenase parent and Mutants Thereof

| | Relative Enzyme | e.e |
|---|---|---|
| Enzyme | Activity (%) | (%) |
| LcGluDH | 100^{a} | 99.9 |
| LcGluDH-V73C | 25298 ± 12.5 | 99.9 |
| LcGluDH-M91G | 3125.8 ± 10..8 | 99.9 |
| LcGluDH-V73C-M91G | 3986.2 ± 17.5 | 99.9 |

^{a}Under standard conditions, the initial enzyme activity of each phosphinothricin dehydrogenase parent strain is designated as 100%.

### Example 7: E. coli BL21(DE3)/pETDuet-LcGluDH-ADH Transformed 200 mM 4-(Hydroxymethylphosphinyl)-2-Oxobutyric Acid

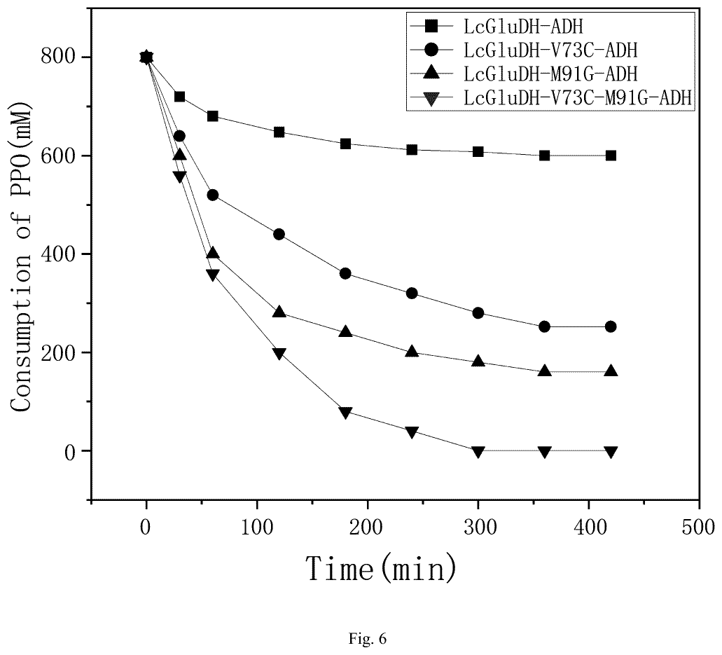

200 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 240 mM, NAD⁺ with a final concentration of 0.4 mM and the wet cells of E. coli BL21(DE3)/pETDuet-LcGluDH-ADH prepared according to the method in Example 3 with a final concentration of 10 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 6), the residue concentration of the substrate was 150 mM, the conversion rate was 25%, the concentration of L-PPT was 48 mM, and the ee value of the product L-phosphinothricin was 99.9%.

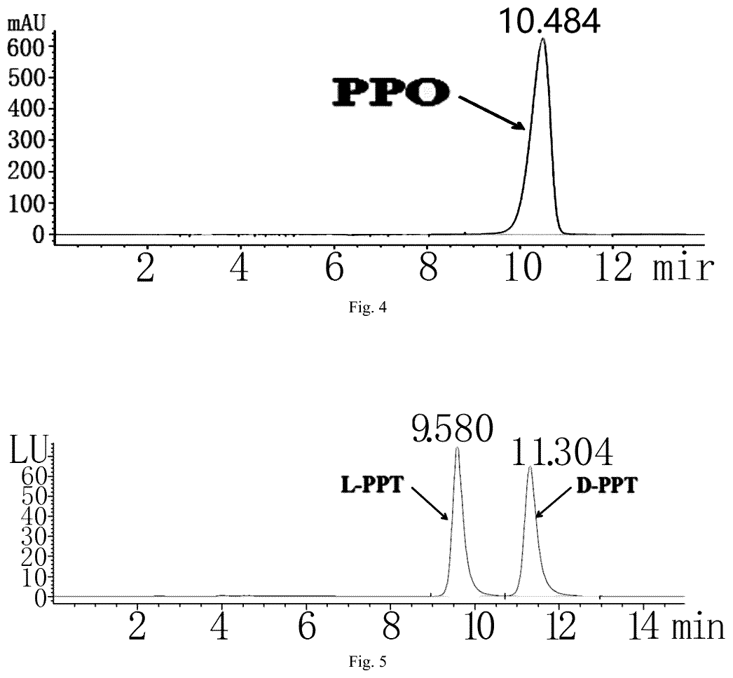

The liquid chromatogram of the 4-(hydroxymethylphosphinyl)-2-oxobutyric acid standard was shown in FIG. 4, and the liquid chromatograms of the D-PPT standard and the L-PPT standard were shown in FIG. 5.

### Example 8: E. coli BL21(DE3)/pETDuet-LcGluDH-V73C-ADH Transformed 200 mM 4-(Hydroxymethylphosphinyl)-2-Oxobutyric Acid

200 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 240 mM, NAD+ with a final concentration of 0.4 mM and the wet cells of E. coli BL21(DE3)/pETDuet-LcGluDH-V73C-ADH prepared according to the method in Example 4 with a final concentration of 10 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 6), the residue concentration of the substrate was 63 mM, the conversion rate was 68.5%, the concentration of L-PPT was 136 mM, and the ee value of the product L-phosphinothricin was 99.9%.

### Example 9: E. coli BL21(DE3)/pETDuet-LcGluDH-M91G-ADH Transformed 200 mM 4-(Hydroxymethylphosphinyl)-2-Oxobutyric Acid

200 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 240 mM, NAD+ with a final concentration of 0.4 mM and the wet cells of E. coli BL21(DE3)/pETDuet-LcGluDH-M91G-ADH prepared according to the method in Example 4 with a final concentration of 10 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 6), the residue concentration of the substrate was 40 mM, the conversion rate of the substrate was 80%, the concentration of L-PPT was 159 mM, and the ee value of the product L-phosphinothricin was 99.9%.

### Example 10: E. coli BL21(DE3)/pETDuet-LcGluDH-V73C-M91G-ADH Transformed 200 mM 4-(Hydroxymethylphosphinyl)-2-Oxobutyric Acid

200 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 240 mM, NAD+ with a final concentration of 0.4 mM and the wet cells of E. coli BL21(DE3)/pETDuet-LcGluDH-V73C-M91G-ADH prepared according to the method in Example 4 with a final concentration of 10 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 6), the residue concentration of the substrate was 0 mM, the conversion rate of the substrate was 100%, the concentration of L-PPT was 199 mM, and the ee value of the product L-phosphinothricin was 99.9%.

### Example 11: E. coli BL21(DE3)/pETDuet-LcGluDH-ADH Transformed 800 mM 4-(Hydroxymethylphosphinyl)-2-Oxobutyric Acid

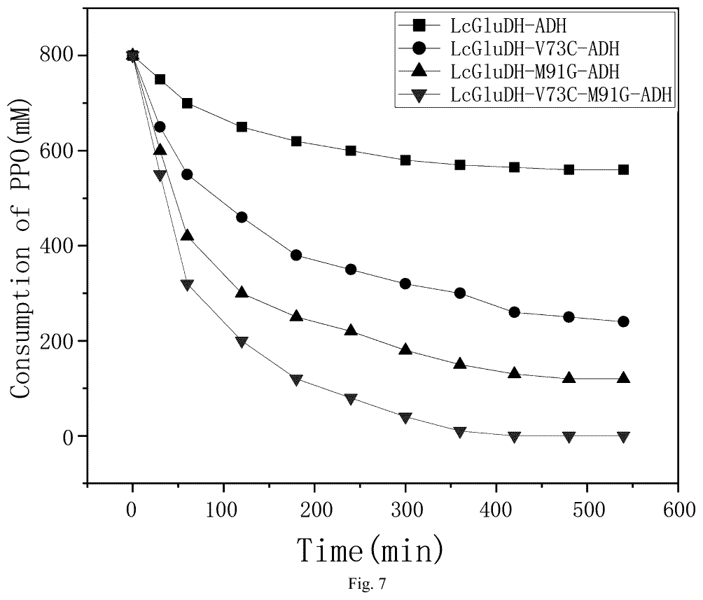

800 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 960 mM, NAD+ with a final concentration of 0.8 mM and the wet cells of E. coli BL21(DE3)/pETDuet-LcGluDH-ADH prepared according to the method in Example 3 with a final concentration of 40 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 7), the residue concentration of the substrate was 560 mM, the conversion rate of the substrate was 30%, the concentration of L-PPT was 239 mM, and the ee value of the product L-phosphinothricin was 99.9%.

### Example 12: E. coli BL21(DE3)/pETDuet-LcGluDH-V73C-ADH Transformed 800 mM 4-(Hydroxymethylphosphinyl)-2-Oxobutyric Acid

800 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 960 mM, NAD+ with a final concentration of 0.8 mM and the wet cells of E. coli BL21(DE3)/pETDuet-LcGluDH-V73C-ADH prepared according to the method in Example 4 with a final concentration of 40 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 7), the residue concentration of the substrate was 240 mM, the conversion rate of the substrate was 70%, the concentration of L-PPT was 559 mM, and the ee value of the product L-phosphinothricin was 99.9%.

### Example 13: E. coli BL21(DE3)/pETDuet-LcGluDH-M91G-ADH Transformed 800 mM 4-(Hydroxymethylphosphinyl)-2-Oxobutyric Acid

800 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 960 mM, NAD+ with a final concentration of 0.8 mM and the wet cells of E. coli BL21(DE3)/pETDuet-LcGluDH-M91G-ADH prepared according to the method in Example 4 with a final concentration of 40 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 7), the residue concentration of the substrate was 120 mM, the conversion rate of the substrate was 85%, the concentration of L-PPT was 680 mM, and the ee value of the product L-phosphinothricin was 99.9%.

### Example 14: E. coli BL21(DE3)/pETDuet-LcGluDH-V73C-M91G-ADH Transformed 800 mM 4-(Hydroxymethylphosphinyl)-2-Oxobutyric Acid

800 mL of 2M 4-(hydroxymethylphosphinyl)-2-oxobutyric acid solution prepared by the method of Example 2 was added into a 3 L common stirring reaction kettle, the pH value of the solution was adjusted to 7.5 with ammonia water, isopropanol with a final concentration of 960 mM, NAD+ with a final concentration of 0.8 mM and the wet cells of E. coli BL21(DE3)/pETDuet-LcGluDH-V73C-M91G-ADH prepared according to the method in Example 4 with a final concentration of 40 g/L were added, the reaction system was added to 2000 mL with a phosphate buffer solution at pH 7.5, then the reaction was conducted at 55° C. and 150 rpm for 15 hours, the reaction solution was subjected to liquid phase detection according to the method of example 4 (FIG. 7), the residue concentration of the substrate was 0 mM, the conversion rate of the substrate was 100%, the concentration of L-PPT was 799 mM, and the ee value of the product L-phosphinothricin was 99.9%.

1. A method for synthesis of L-phosphinothricin, comprising the following steps:
forming a reaction system by using the wet cells obtained by fermentative cultivation of an engineered strain of recombinant Escherichia coli that co-express the gene encoding phosphinothricin dehydrogenase and the gene encoding alcohol dehydrogenase, or that co-express the gene encoding a phosphinothricin dehydrogenase mutant and the gene encoding alcohol dehydrogenase as a catalyst, using 4-(hydroxymethylphosphinyl)-2-oxobutyric acid as a substrate, adding isopropanol and NAD+, and using a buffer solution with a pH of 6-8 as the reaction medium, conducting the conversion reaction at 40-60° C. and 100-200 rpm, and subjecting the reaction solution to separation and purification to obtain L-phosphinothricin; wherein the phosphinothricin dehydrogenase mutant is obtained by single or double mutation at position 73 or 91 of the phosphinothricin dehydrogenase with the amino acid sequence shown as SEQ ID NO.2.

2. The method for synthesis of L-phosphinothricin according to claim 1, wherein the preparation of 4-(hydroxymethylphosphinyl)-2-oxobutyric acid comprises:
(1) synthesizing 3-(methylethoxyphosphinyl) ethyl propionate by addition reaction from diethoxymethylphosphine and acrylic acid under the conditions of temperature 100-150° C. and pressure 0.5-5 MPa;
(2) conducting the condensation reaction of 3-(methylethoxyphosphinyl) ethyl propionate with sodium ethoxide and diethyl oxalate under the conditions of temperature 40-80° C. and pressure 1-5 MPa, then adjusting the pH value of the reaction solution to 6-8, subjecting the reaction solution to heating and hydrolysis reaction at 50-70° C., rectifying the reaction liquid to recover the byproduct ethanol, then filtering to remove sodium chloride, and taking the filtrate to obtain 4-(hydroxymethylphosphinyl)-2-oxobutyric acid.

3. The method for synthesis of L-phosphinothricin according to claim 2, wherein the volume ratio of acrylic acid to diethoxymethylphosphine described in step (1) is 1-5:1.

4. The method for synthesis of L-phosphinothricin according to claim 2, wherein in step (2), the condensation reaction is performed for 8 h at a temperature of 60° C. and a pressure of 1.5 MPa; and the temperature of the hydrolysis reaction is 60° C.

5. The method for synthesis of L-phosphinothricin according to claim 2, wherein in step (2), the volume of 3-(methylethoxyphosphinyl) ethyl propionate is 20-50 L/kg in terms of the mass of sodium ethylate; and the mass ratio of sodium ethylate to diethyl oxalate is 1:1-3.

6. The method for synthesis of L-phosphinothricin according to claim 1, wherein in the reaction system, the final concentration of the wet cells added to the reaction system is 10-40 g/L, and the final concentration of the substrate added to the reaction system is 200-800 mM; the final concentration of isopropanol added to the reaction system is 200-1000 mM; and the final concentration of NAD+added to the reaction system is 0.4-0.8 mM.

7. The method for synthesis of L-phosphinothricin according to claim 1, wherein the phosphinothricin dehydrogenase mutant is obtained by mutating the amino acid sequence shown as SEQ ID NO. 2 in one of the following ways: (1) mutating valine at position 73 into cysteine; (2) mutating methionine at position 91 into glycine; or (3) mutating valine at position 73 into cysteine and methionine at position 91 into glycine.

8. The method for synthesis of L-phosphinothricin according to claim 1, wherein the preparation method of the catalyst comprises: inoculating the engineered strain of recombinant Escherichia coli co-expressing the gene encoding phosphinothricin dehydrogenase and the gene encoding alcohol dehydrogenase, or co-expressing the gene encoding the phosphinothricin dehydrogenase mutant and the gene encoding alcohol dehydrogenase into an LB liquid culture medium containing ampicillin with a final concentration of 50 μg/ml, culturing it at 37° C. for 8 hours, then inoculating it into a fresh LB liquid culture medium containing ampicillin with a final concentration of 50 μg/ml, culturing it at 37° C. and 180 r/min for 2 hours, then adding IPTG with a final concentration of 0.1 mM into the culture solution, culturing it at 18° C. for 14 hours, and centrifuging it at 4° C. and 8000 rpm for 10 minutes to obtain corresponding wet cells.

9. The method for synthesis of L-phosphinothricin according to claim 1, wherein the amino acid sequence of the alcohol dehydrogenase is as shown in SEQ ID NO. 4.

10. A phosphinothricin dehydrogenase mutant for synthesis of L-phosphinothricin by the method of claim 1, wherein the phosphinothricin dehydrogenase mutant is obtained by performing single mutation or double mutation at position 73 or 91 of the glufosinate dehydrogenase shown in SEQ ID NO. 2.

11. The phosphinothricin dehydrogenase mutant for synthesis of L-phosphinothricin of claim 10, wherein the mutant is obtained by mutating the amino acid sequence shown as SEQ ID NO. 2 in one of the following ways: (1) mutating valine at position 73 into cysteine; (2) mutating methionine at position 91 into glycine; or (3) mutating valine at position 73 into cysteine and methionine at position 91 into glycine.
